(19) United States
(12) Patent Application Publication
(10) Pub. No.: US 2025/0331777 A1
(43) Pub. Date: Oct. 30, 2025

(54) SENSOR SUSPENSION SYSTEM FOR SUPINE CO2 MONITORING

(71) Applicant: Arizona Board of Regents on Behalf of the University of Arizona, Tucson, AZ (US)

(72) Inventors: Yijie Chen, Tucson, AZ (US); Md Tariqul Islam, Tucson, AZ (US); Young-Jun Son, Tucson, AZ (US); Esther M. Sternberg, Tucson, AZ (US); Christopher J. Morton, Tucson, AZ (US)

(21) Appl. No.: 18/880,002

(22) PCT Filed: Jun. 30, 2023
(86) PCT No.: PCT/US2023/069484

§ 371 Date: Dec. 30, 2024

Related U.S. Application Data
(60) Provisional application No. 63/357,069, filed on Jun. 30, 2022.

(51) Int. Cl. A61B 5/00

(52) U.S. Cl. CPC A61B 5/6887; A61B 5/4812; A61B 5/7253; A61B 5/7267; A61B 2562/046; A61B 2562/16

(57) ABSTRACT

Various examples are provided related to suspension of sensors for monitoring of gases. In one example, a sensor suspension system includes sensor holders including a channel attachment and a sensor frame to support a sensor at ends of a support arm; and a support frame including channels that can engage with the channel attachment to support the sensor holder from the support frame. Positioning of each sensor holder can be adjusted about the support frame by sliding the channel attachment within the channels of the support frame. In another example, a method includes positioning a sensor suspension system over a face of a subject; adjusting positioning of one or more CO₂ sensor supported by the sensor suspension system; and obtaining CO₂ concentration readings from sensors supported by the sensor suspension system.

### CROSS REFERENCE TO RELATED APPLICATIONS

This application claims priority to, and the benefit of, co-pending U.S. provisional application entitled “Sensor Suspension System for Supine CO₂ Monitoring” having Ser. No. 63/357,069, filed Jun. 30, 2022, which is hereby incorporated by reference in its entirety.

### BACKGROUND

Acute respiratory distress syndrome (ARDS) is a major complication in patients with severe COVID-19 pulmonary disease, which can manifest shortly after the onset of difficulty breathing. As ARDS onset can occur very quickly after the appearance of mild respiratory symptoms of COVID-19, it is important to have ways to monitor and quickly identify respiratory decline before it reaches a critical level. Current evaluation of respiratory distress and failure utilizes cumbersome and relatively invasive pulmonary function tests such as, e.g., spirometry, lung volume, and lung diffusion capacity. However, patients with severe ARDS may be unconscious or too weak to effectively perform these tests, and likely cannot be transported to testing equipment.

### SUMMARY

Aspects of the present disclosure are related to suspension of sensors for monitoring of gases. In one aspect, among others, a sensor suspension system comprises a plurality of sensor holders comprising a channel attachment at a proximal end of a support arm and a sensor frame at a distal end of the support arm, the sensor frame configured to support at least one sensor; and a support frame comprising channels distributed about the support frame, the channel attachment of each of the plurality of sensor holders configured to engage with the channels to support that sensor holder from the support frame, where positioning of each sensor holder is adjustable about the support frame by sliding the channel attachment within the channels of the support frame. In one or more aspects, the channel attachment can comprise a sliding block configured for insertion and movement within the channels. The sensor frame can be substantially parallel to the support arm. The sensor frame can be configured to support a sensor. The sensor can be a CO₂ sensor. The plurality of sensor holders and support frame can be transparent. The plurality of sensor holders and support frame can be fabricated from a resin.

In various aspects, at least a portion of the channels can be distributed about at least a perimeter of the support frame. At least a portion of the channels can be distributed along spokes of the support frame. The perimeter can be an outer ring comprising a continuous channel extending around the outer ring. The outer ring can comprise one or more opening in the outer ring. The one or more opening can extend through the outer ring to the continuous channel The one or more opening can provide visual access to the channel attachment of a sensor holder engaged with the continuous channel. The support frame can comprise spokes extending from a central mounting structure to the outer ring. The spokes can comprise radial channels extending along a length of the spoke to the continuous channel around the outer ring. The spokes can comprise an opening extending through the spoke to the radial channel. The opening can provide visual access to the channel attachment of a sensor holder engaged with the radial channel. The support frame can comprise a plurality of sector pieces, each sensor piece comprising at least one spoke and a portion of the outer ring. Adjacent sector pieces can be coupled together via bolt structures. The support frame can be supported by the central mounting structure.

In another aspect, a method comprises positioning a sensor suspension system over a face of a subject, the sensor suspension system comprising: a plurality of sensor holders comprising a channel attachment at a proximal end of a support arm and a sensor frame at a distal end of the support arm, the sensor frame supporting at least one sensor; and a support frame comprising channels distributed about the support frame, the channel attachment of each of the plurality of sensor holders engaged with the channels to support that sensor holder from the support frame, where positioning of each sensor holder is adjustable about the support frame by sliding the channel attachment within the channels of the support frame; adjusting positioning of one or more CO₂ sensor supported by the plurality of sensor holders, the one or more CO₂ sensor located at a distance from the subject's face; and obtaining CO₂ concentration readings from sensors supported by the sensor suspension system. The one or more CO₂ sensor can be located at a distance less than 20 cm from the subject's face or at a distance of about 15 cm or less from the subject's face. In various aspects, the method can comprise correlating sequences of CO₂ concentration readings with sleep stages identified with polysomnography; training a dynamic data wrapping (DTW) model based upon the correlated sequences of CO₂ concentration readings; and identifying a sleep stage of a subsequent sequence of CO₂ concentration readings using the trained DTW model. Training of the DTW model can be based upon patterns extracted from the correlated sequences of CO₂ concentration readings using a shapelet transformation.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a perspective view illustrating an example of a sensor suspension system comprising a support frame suspending sensor holders, in accordance with various embodiments of the present disclosure.

FIGS. 2A-2C are top, side, and bottom views of the sensor suspension system of FIG. 1, in accordance with various embodiments of the present disclosure.

FIGS. 3A and 3B illustrate examples of a sector piece of the support frame and a sensor holder of the sensor suspension system of FIG. 1

FIG. 4 illustrates examples of CO₂ concentration readings obtained using the sensor suspension system of FIG. 1 at different distances from the subject's face, in accordance with various embodiments of the present disclosure.

FIG. 5 illustrates an example of dynamic data wrapping (DTW), in accordance with various embodiments of the present disclosure.

FIG. 6 illustrates an example of a shapelet extraction algorithm that can be used for for CO₂ pattern extraction), in accordance with various embodiments of the present disclosure.

FIG. 7 illustrates examples of CO₂ disturbance patterns extracted for different sleep stages, in accordance with various embodiments of the present disclosure.

### DETAILED DESCRIPTION

Disclosed herein are various examples related to suspension of sensors for monitoring of gases. For example, a sensor suspension system is disclosed that can be used for CO₂ monitoring. Reference will now be made in detail to the description of the embodiments as illustrated in the drawings, wherein like reference numbers indicate like parts throughout the several views.

Exhaled breath analysis may be used to diagnose ARDS and other lung diseases. While most prior devices measure exhaled volatile organic compounds, measurement of exhaled CO₂ can an effective method to measure lung function. However, the devices for collection of exhaled respiratory gases are invasive or cumbersome, some being applicable only in intubated patients, and others requiring mouth devices for gas collection. Methods for measuring respiratory rate include a nasal cannula and a thermistor that measures the temperature change between the inhaled air and exhaled respiratory gases. Most clinical approaches for diagnosis of ARDS require patients' active participation to achieve diagnostic test accuracy. Lung imaging typically needs transport to radiology which exposes healthcare providers to possible viral exposure. Moreover, transport for imaging as well as invasive testing in patients with severe ARDS increases the risk of secondary bacterial infection to these patients, a known contributor to mortality.

In this disclosure, a remote detection methodology using a multi-sensor respiratory monitoring system for continual measurement of respiratory parameters of human exhaled respiratory gases is described. Use of a sensor suspension system allows for monitoring without patient contact. It has been shown that during exhalation a “CO₂ bubble” around a person's face has CO₂ concentrations ranging from, e.g., 400 to 1200 ppm. Current CO₂ detection sensors utilize infrared spectral measurement to accurately measure CO₂ levels. These sensors have varying ranges of sensitivity, some more sensitive at lower ranges equivalent to room air (e.g., 400-2000 ppm CO₂), and others more sensitive at higher ranges for use in industrial settings (e.g., >10,000 ppm CO₂). To measure the exhaled CO₂ bubble accurately and sensitively, an arrangement of sensors close to the mouth can be used.

Current non-invasive measurements of the CO₂ concentration in human respiratory gas mainly rely on a respiratory mask which is unable to detect the natural CO₂ accumulation of both the respiration and the gas distribution in a room environment. Other methods such as wall-mounted environment CO₂ sensor are limited to the distance and cannot provide a 3-dimensional CO₂ distribution around the human face. Therefore, a non-invasive, canopy shape, stereoscopic CO₂ monitoring system is proposed to measure the CO₂ distribution around the human face. A multi-CO₂-mini-sensor based monitoring system has been developed that can measure the CO₂ concentration of human respiratory gas around subject's face while the subject is in a supine position. The system can support further analysis of human's physical conditions, such as sleep stage and sleep quality according to the collected data.

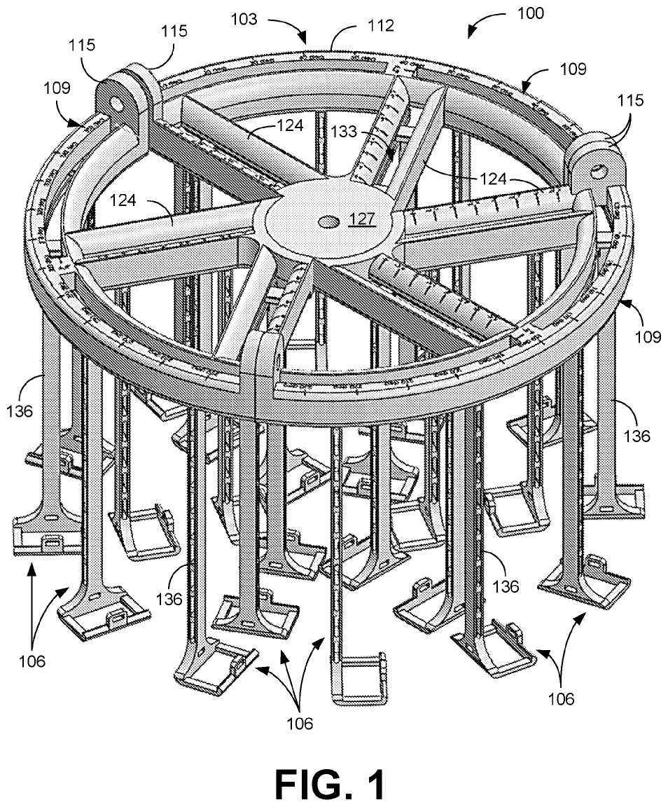

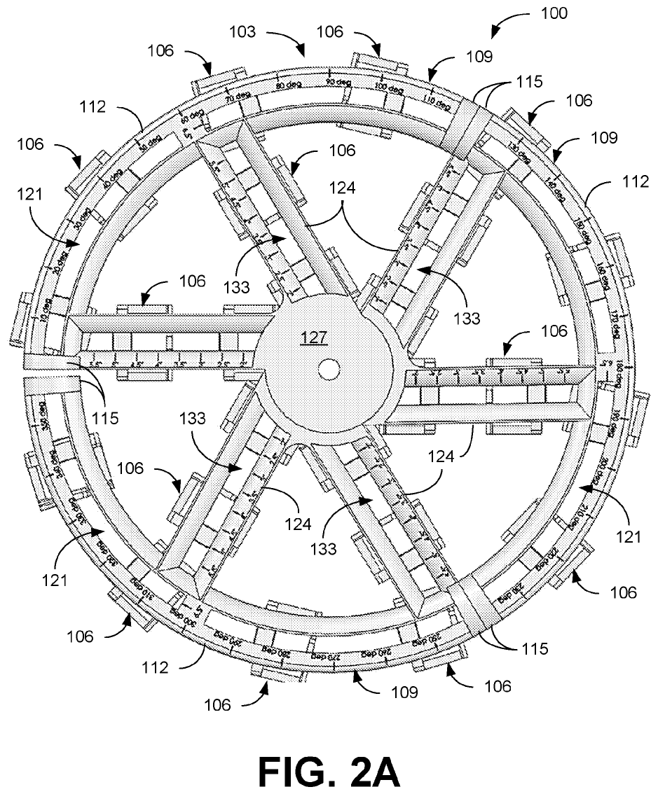

Referring to FIG. 1, shown is an example of a sensor suspension system 100. The sensor suspension system 100 comprises a round support frame 103 and multiple sensor holders 106 suspended from the support frame 103. While the support from is shown in a round or circular configuration, other geometric configurations (e.g., triangular, square, pentagonal, hexagonal, octagonal, etc.) are also possible. FIG. 2A is a top view of the sensor suspension system 100 illustrating the support frame 103. FIG. 2B is a side view of the sensor suspension system 100 illustrating the sensor holders 106 suspended from the support frame 103. FIG. 2C is a bottom view of the sensor suspension system 100 illustrating a distribution of the sensor holders 106 below the support frame 103.

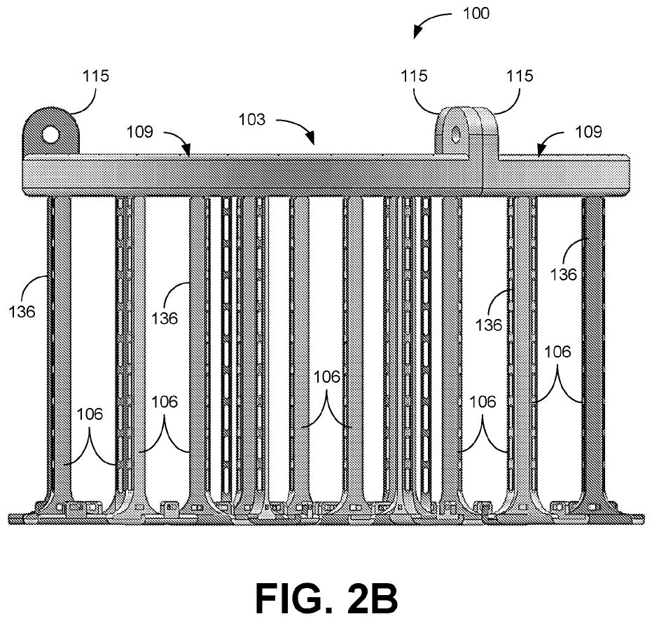

The round support frame 103 includes three sector pieces 109, each of 120 degrees. FIG. 3A illustrates an example of a sector piece 109. The sector piece 109 includes an outer ring 112 that can serve as a curved slideway for sensor holders 106 supported by the outer ring 112. Each sector piece 109 can include a raised bolt structure 115 at both ends for fixation of the sector pieces 109. An appropriate fastener (e.g., bolt and nut, screw, etc.) can be used to fasten the sector pieces 109 together by the bolt structures 115. As shown in FIG. 3A, the outer ring 112 of the sector piece 109 can include a channel 118 configured to receive an end of one or more sensor holders 106. The sensor holders 106 can be slide along the channel 118 to a desired position along the outer ring 112.

As shown in FIG. 2A, the outer ring 112 can include one or more opening 121 that extends through the outer ring 112 to the channel 118 to allow the position of a sensor holder 106 in the channel 118 to be visible through the sector piece 109. The outer ring 112 can include markings or scales on the top surface of the round frame to facilitate and confirm the position and/or angle of the suspended sensor holders 106. Two radial spokes 124 can connect the outer ring 112 to an inner plate 127 of the sector piece 109. Each spoke 124 can include a channel 130 extending from the channel 118 in the outer ring 112 to the inner plate 127. Sensor holders 106 can enter the channel 130 through the channel 118 of the outer ring 112 as shown in FIG. 2C. The spokes 124 can also include one or more opening 133 (FIG. 2A) that extends through the spoke 124 to the channel 130 to allow the position of a sensor holder 106 in the channel 130 to be visible through the sector piece 109. The spoke 124 can include markings or scales on the top surface to facilitate and confirm the position of the suspended sensor holders 106. The inner plate 127 can include a hole or opening in the center for frame assembly with, e.g., a long rod. The inner plate 127 can have a height equal to about ⅓ of the height of the outer ring 112, which allows the inner plates 127 of three sectors to create a three-ply mounting structure with one bottom plate, one middle plate and one top plate.

FIG. 3B illustrates an example of a sensor holder 106 including a support arm 136 extending between a proximal (or top) end and a distal (or bottom) end. The proximal or top end of the support arm 136 can include a channel attachment such as, e.g., a sliding block 139 or other appropriate engagement arrangement configured for insertion and movement in the channels 118 and 130 of the outer ring 112 and radial spokes 124. The distal or bottom end of the support arm 136 comprises a sensor frame 142 configured to support a monitoring sensor (e.g., a CO₂ sensor). In the example of FIG. 3B, the sensor frame 142 comprises a rectangular support frame which fits the size of a CO₂ sensor (e.g., a S8 CO₂ mini sensor) and a slot located above the sensor to allow the sensor to be secured to the framework by, e.g., a clip, pin, or other appropriate fastener. The length of the support arm 136 can be fixed or adjustable. In the example shown in FIGS. 1 and 2B, the length of the support arm 136 is the same for all sensor holders 106, but different support arm lengths may be used to vary the position of the monitoring sensors supported by the sensor suspension system 100.

The sensor holders 106 can be configured to support a wide range of sensors including CO₂ sensors, thermal sensors, etc. For example, mini-CO₂ sensors can be supported by the sensor frame 142 of the sensor holders 106 for sampling CO₂ concentrations in a range of 0-50000 ppm, with 1 reading per second, and a ±70 ppm accuracy. The sensors can be controlled by processing circuitry including a processor and memory, and which can include circuitry for transmission of the sampled data to another device for processing and analysis. For example, each CO₂ sensor can be controlled by a raspberry pi, and the raspberry pi can transmit real-time data points or other information wirelessly to a remote computing device such as, e.g., a laptop, tablet or smartphone via a wireless communication link (e.g., Bluetooth® or Wi-Fi). The transmitted information can include a sensor identifier that can be used to correlate the data with the position of the sensor.

The sensor suspension system 100 can be fabricated from resin, plastic, metal or other appropriate material. The initial suspension system 100 was developed using, e.g., Solid Work software and printed out using a 3D printer (e.g., a FormLabs 3L 3D printer). The whole structure of the sensor suspension system 100 can be optimized to reduce the disturbance to the areo-dynamic of the CO₂ bubble with multiple hollowing designs. Considering the potential mental pressure on a human subject laying under the sensor suspension system, it was printed with a transparent resin to reduce the oppression and negative feeling of the subject.

Utilizing the sensor suspension system 100, three-dimensional CO₂ bubble visualization can be achieved with a continuous estimation of the CO₂ concentration inside the bubble based on real-time data. The data analysis from one eight-hour sleep study using the primary suspension system showed a correlation between CO₂ bubble shape, CO₂ concentration inside the bubble and the sleep stages (awake, light sleep, deep sleep and REM) of the human subject. The CO₂ concentration level and the CO₂ bubble shape can reflect the sleep quality of human subjects as well.

Studies can be carried out (e.g., formal sleep experiments in a sleep lab) with a sensor suspension system 100 positioned above the face of human subject. The subject can evaluate the comfort level of the suspension system to determine whether the system is user friendly. Data from multiple human subjects can be collected using the sensor support system 100 and analyzed to validate the performance of the sensor support system 100. Algorithms can be used to calculate the sleep stage based on the real-time CO₂ concentration readings. For example, sleep-tech data from Sleepware G3 such as ECG, CanFlow, TFlow, SpO2, Mscore and ECG-based sleep stage can serve as a ground truth to verify the results from the data obtained using the sensor suspension system 100.

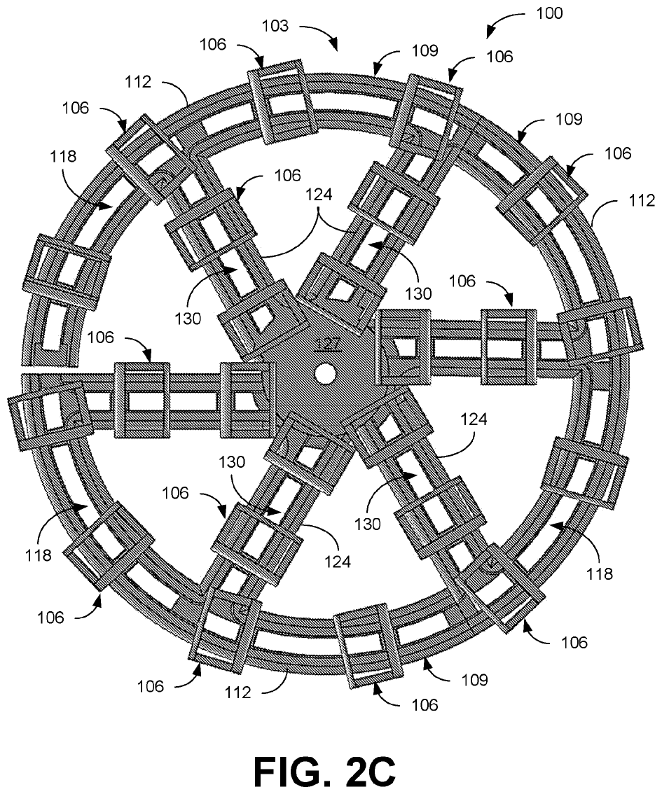

The sensor suspension system 100 provides non-invasive monitoring without the need for direct contact with the subject. It can indicate the interaction of indoor air quality with the micro-space around the subject's face. The human respiration condition reflected by the sensor system 100 can be more sensitive and faster. FIG. 4 illustrates examples of CO₂ concentration readings obtained using the sensor suspension system 100 at different distances from the subject's face. While the sensors can be located at a range of distances over the subject as shown in FIG. 4, a distance of less than 20 cm or about 15 cm or less can sense higher concentrations of CO₂. Comparing preliminary experiment results to other existing works which only monitor the environment's CO₂ concentration, the sensor suspension system 100 exhibits a more accurate estimation of potential human cognitive performance impairment. Additionally, the experiment results showed that the environment CO₂ sensor is easily disturbed by the ventilation system rather than the human subject's respiration or a physiological status change.

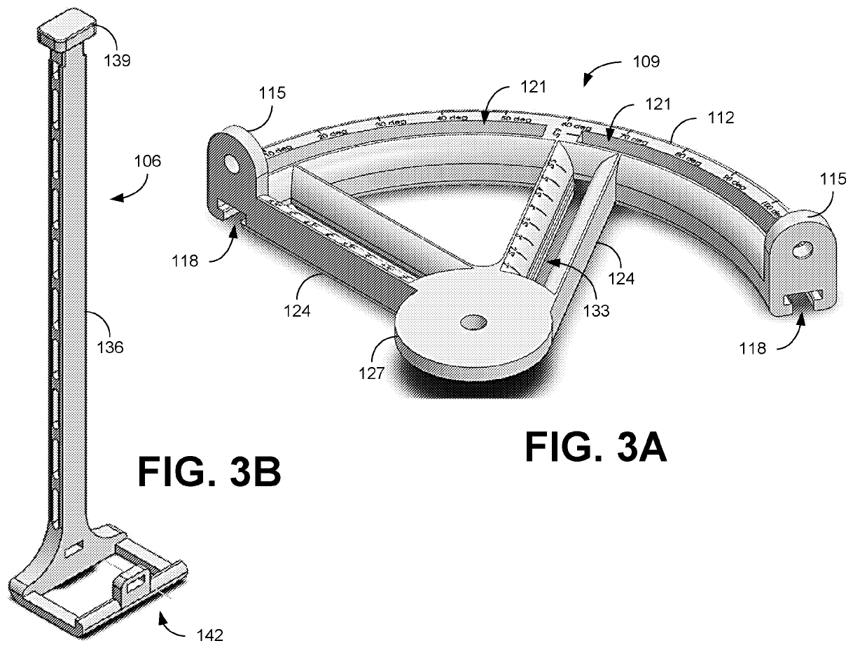

The sensor suspension system 100 also allows analysis of the sleep quality and sleep stage of the human subject with dynamic data wrapping (DTW) and machine learning methods. DTW is a technique that can be used to measure the similarity between two temporal sequences, even if they have different lengths or speeds. FIG. 5 is a visualization of the DTW technique. For the data preprocessing, noise removal, filtering, resampling, and data smoothing can be applied to the CO₂ concentration sensor readings. With the sleep stage labels obtained from a polysomnography (PSG) system, a DTW model can be developed that can be used to learn the relationship between the CO₂ concentration features and sleep stages via templates constructed by aligning the CO₂ concentration sequences of each sleep stage using DTW. For the testing stage, the DTW algorithm can measure the similarity between the features of each testing sample and the reference templates for different sleep stages. The predicted sleep stage can be identified as the one with the highest similarity score.

In one experiment, the sensor suspension system 100 was set to be 15 cm away from the subject for monitoring and sleep-related information was collected from the PSG system. During the 8-hour experiment period, only data collected when the subject was in the supine position was used for analysis. For the data segmentation, CO₂ concentration readings were divided into 3-min periods, giving 89 training samples in total.

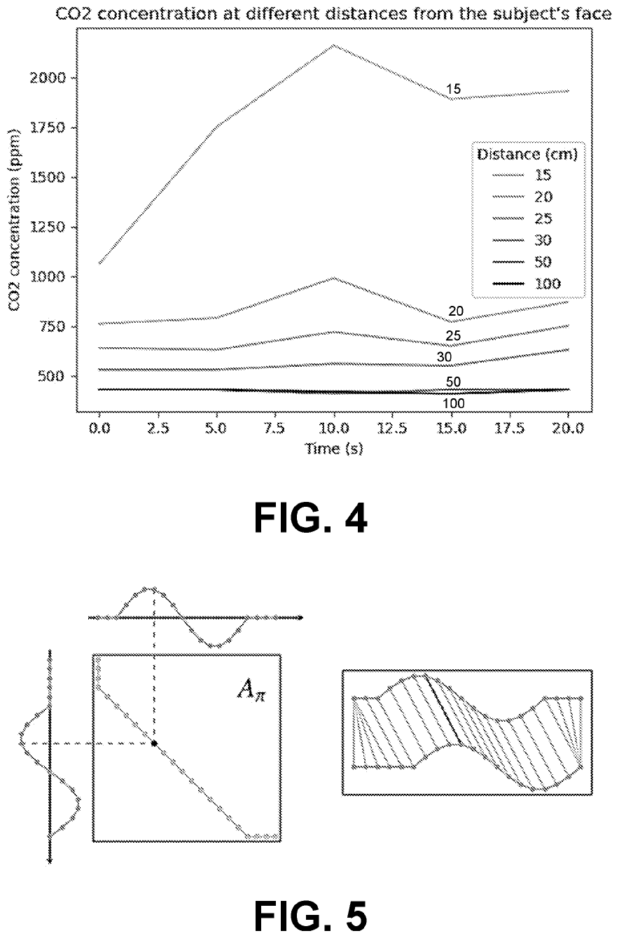

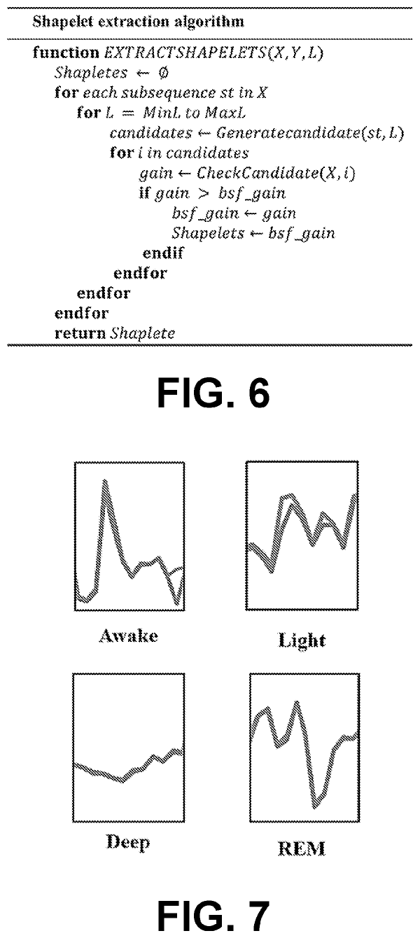

A shapelet transformation can be used in conjunction with the DTW methodology to identify local patterns or subsequences that are discriminative and representative of the current sleep stage. FIG. 6 shows pseudo codes illustrating the shapelet extraction algorithm used with the data training for CO₂ pattern extraction. Examples of CO₂ disturbance patterns extracted for different sleep stages (e.g., awake and light, deep and REM sleep stages) are illustrated in FIG. 7. The results show that the prediction accuracy for sleep stage classification is 72.4%. The CO₂ disturbance patterns can be used to train machine learning to identify sleep stages. The trained machine learning can be used with DWT for identification during subsequent monitoring.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y” includes “about ‘x’ to about ‘y’”.

1. A sensor suspension system, comprising:
a plurality of sensor holders comprising a channel attachment at a proximal end of a support arm and a sensor frame at a distal end of the support arm, the sensor frame configured to support at least one sensor; and
a support frame comprising channels distributed about the support frame, the channel attachment of each of the plurality of sensor holders configured to engage with the channels to support that sensor holder from the support frame, where positioning of each sensor holder is adjustable about the support frame by sliding the channel attachment within the channels of the support frame.

2. The sensor suspension system of claim 1, wherein at least a portion of the channels is distributed about at least a perimeter of the support frame.

3. The sensor suspension system of claim 1, wherein at least a portion of the channels are distributed along spokes of the support frame.

4. The sensor suspension system of claim 2, wherein the perimeter is an outer ring comprising a continuous channel extending around the outer ring.

5. The sensor suspension system of claim 4, wherein the outer ring comprises one or more opening in the outer ring, the one or more opening extending through the outer ring to the continuous channel, the one or more opening providing visual access to the channel attachment of a sensor holder engaged with the continuous channel.

6. The sensor suspension system of claim 4, wherein the support frame comprises spokes extending from a central mounting structure to the outer ring, the spokes comprising radial channels extending along a length of the spoke to the continuous channel around the outer ring.

7. The sensor suspension system of claim 6, wherein the spokes comprise an opening extending through the spoke to the radial channel, the opening providing visual access to the channel attachment of a sensor holder engaged with the radial channel.

8. The sensor suspension system of claim 6, wherein the support frame comprises a plurality of sector pieces, each sensor piece comprising at least one spoke and a portion of the outer ring.

9. The sensor suspension system of claim 8, wherein adjacent sector pieces are coupled together via bolt structures.

10. The sensor suspension system of claim 6, wherein the support frame is supported by the central mounting structure.

11. The sensor suspension system of claim 1, wherein the channel attachment comprises a sliding block configured for insertion and movement within the channels.

12. The sensor suspension system of claim 1, wherein the sensor frame is substantially parallel to the support arm.

13. The sensor suspension system of claim 1, wherein the sensor frame is configured to support a sensor.

14. The sensor suspension system of claim 13, wherein the sensor is a CO₂ sensor.

15. The sensor suspension system of claim 1, wherein the plurality of sensor holders and support frame are transparent.

16. The sensor suspension system of claim 1, wherein the plurality of sensor holders and support frame are fabricated from a resin.

17. A method, comprising:
positioning a sensor suspension system over a face of a subject, the sensor suspension system comprising:
a plurality of sensor holders comprising a channel attachment at a proximal end of a support arm and a sensor frame at a distal end of the support arm, the sensor frame supporting at least one sensor; and
a support frame comprising channels distributed about the support frame, the channel attachment of each of the plurality of sensor holders engaged with the channels to support that sensor holder from the support frame, where positioning of each sensor holder is adjustable about the support frame by sliding the channel attachment within the channels of the support frame;
adjusting positioning of one or more CO₂ sensor supported by the plurality of sensor holders, the one or more CO₂ sensor located at a distance of less than 20 cm from the subject's face; and
obtaining CO₂ concentration readings from sensors supported by the sensor suspension system.

18. The method of claim 17, comprising correlating sequences of CO₂ concentration readings with sleep stages identified with polysomnography;
training a dynamic data wrapping (DTW) model based upon the correlated sequences of CO₂ concentration readings; and
identifying a sleep stage of a subsequent sequence of CO₂ concentration readings using the trained DTW model.

19. The method of claim 18, wherein training of the DTW model is based upon patterns extracted from the correlated sequences of CO₂ concentration readings using a shapelet transformation.

20. The method of claim 17, wherein the one or more CO₂ sensor is located at a distance of about 15 cm or less from the subject's face.
